(19) United States
(12) Patent Application Publication
(10) Pub. No.: US 2026/0351347 A1
(43) Pub. Date: Nov. 5, 2026

(54) METHOD AND APPARATUS FOR DNA ENCODING AND DECODING

(71) Applicant: Keun Woo LEE, Yongin-si, Gyeonggi-do (KR)

(72) Inventor: Keun Woo LEE, Yongin-si, Gyeonggi-do (KR)

(21) Appl. No.: 19/118,771

(22) PCT Filed: Sep. 19, 2023
(86) PCT No.: PCT/KR2023/014125

§ 371 Date: Apr. 6, 2025

(30) Foreign Application Priority Data
Oct. 6, 2022 (KR) 10-2022-0127750
Oct. 11, 2022 (KR) 10-2022-0129385
Oct. 24, 2022 (KR) 10-2022-0137061
Oct. 26, 2022 (KR) 10-2022-0138893

(51) Int. Cl. G16B 30/20; G16B 50/50

(52) U.S. Cl. CPC G16B 30/20; G16B 50/50

(57) ABSTRACT

A DNA encoding method includes: (a) a step in which a processor performs a primary conversion of binary data into a base sequence; (b) a step in which the processor finds a point where the same base is repeated within the primarily converted base sequence; and (c) a step in which the processor performs a secondary conversion into a base sequence to which a dummy base is added at the point where the same base is repeated.

### TECHNICAL FIELD

The present invention relates to DNA molecule-based information storage technology, and more particularly, to encoding techniques for DNA synthesis and decoding techniques for data recovery.

### BACKGROUND ART

This application claims priority based on Korean Patent Application No. 10-2022-0127750 filed on Oct. 6, 2022, No. 10-2022-0129385 filed on Oct. 11, 2022, No. 10-2022-0137061 filed on Oct. 24, 2022, and No. 10-2022-0138893 filed on Oct. 26, 2022, in the Republic of Korea. The entire contents disclosed in the specifications and drawings of the aforementioned applications are incorporated herein by reference.

The information described in this section is provided merely for background purposes regarding the embodiments disclosed in the present specification and is not necessarily to be considered as prior art.

Magnetic tape, which is widely used as a long-term storage medium, has a data retention lifespan of approximately 10 years, thereby requiring continuous maintenance and management costs. In the case of semiconductor-based storage devices, hard disk drives (HDDs) and solid-state drives (SSDs) are representative examples. The lifespan of an HDD is typically around 5 years; however, when used in environments where data access occurs less than once per quarter, the lifespan may extend to approximately 10 years. Nevertheless, HDDs are highly susceptible to physical shock and have limitations in terms of maximum storage capacity. In the case of SSDs, while they exhibit superior resistance to physical shock, their lifespan is relatively shorter compared to HDDs. In recent years, the explosive increase in data production has exceeded the capacity of existing storage media, resulting in system overload. Moreover, conventional data storage devices have reached their limits in terms of data storage density, thereby creating a need for a new type of storage technology.

Among the various attempts to develop new storage media, efforts have been made to utilize DNA as a novel storage medium. When DNA is used as a storage medium, it can overcome the limitations of conventional storage media in terms of data storage density and can stably preserve information over long periods, even under physical shock.

As is well known, DNA is contained within the cell, the smallest unit of a living organism, and carries all genetic information. Based on the information encoded within DNA, all living organisms grow and function as if they were programmed. In the case of humans, the DNA contained within a single cell consists of approximately 3 billion base pairs, and the total decoded genetic information is estimated to be around 1 terabyte in size. Furthermore, a single cell contains two strands of DNA, each having a width of approximately 2 nanometers and a length of approximately 3 meters. Therefore, DNA, as a next-generation bio-storage medium capable of theoretically storing more than an exabyte (1018 bytes) of data, is highly suitable as a biomaterial for ultra-dense information storage. In addition, DNA offers a storage lifespan of over 1,000 years and is expected to enable low-cost data storage.

FIG. 1 is a conceptual diagram illustrating information storage using DNA base sequences.

Referring to FIG. 1, the binary data to be stored is encoded using the nucleotides A (adenine), T (thymine), G (guanine), and C (cytosine). DNA is synthesized according to the encoded base sequence, and the synthesized DNA molecules are stored. Thereafter, the stored DNA molecules are selected through retrieval, the base sequences of the selected DNA molecules are analyzed, and the analyzed sequences are decoded into binary data.

### DETAILED DESCRIPTION OF THE INVENTION

### Technical Problem

An object of the present specification is to provide a DNA encoding method and apparatus that reduce the possibility of errors during DNA synthesis and reconstruction.

An object of the present specification is to provide a method and apparatus for mixing and reconstructing binary data, which reduces the likelihood of errors during DNA synthesis and restoration.

An object of the present specification is to provide a DNA encoding method and apparatus capable of enhancing the structural stability of synthesized DNA.

The present specification is not limited to the above-mentioned objects, and other objects not explicitly stated will be clearly understood by those of ordinary skill in the art from the following description.

### Solution to Problem

To solve the above-described problem, a DNA encoding method according to the present specification may include: (a) a step in which a processor performs a first conversion of binary data into a base sequence; (b) a step in which the processor identifies positions within the first-converted base sequence where identical bases are consecutively repeated; and (c) a step in which the processor performs a second conversion by adding dummy bases at the positions where the identical bases are repeated.

According to one embodiment of the present specification, the step (b) may be a step in which the processor detects a point where a predetermined number of identical bases are repeated.

According to an embodiment of the present specification, the step (b) may be a step in which the processor detects a point at which the same base is consecutively repeated, based on a predetermined number of repetitions set for each base.

According to one embodiment of the present specification, the step (b) may include: (b-1) a step of generating, by the processor, a frequency table for repeated bases within the first-converted base sequence; (b-2) a step of determining, by the processor, the number of consecutive identical bases at which dummy bases need to be added, using the generated frequency table; and (b-3) a step of identifying, by the processor, positions in the first-converted base sequence where identical nucleotides are consecutively repeated, based on the number determined in step (b-2).

According to an embodiment of the present specification, the step (b-2) may be a step in which the processor determines the number of repeated identical bases requiring dummy bases addition, based on an average value of frequency.

According to an embodiment of the present specification, the step (b-2) may be a step in which the processor determines, for each base, the number of repeated identical bases requiring dummy bases addition, based on an average frequency value for each According to an embodiment of the present specification, the step (c) may be a step in which the processor performs a secondary conversion by adding dummy bases having at least one predetermined sequence.

According to an embodiment of the present specification, the step (c) may be a step in which the processor performs a secondary conversion by adding, as dummy bases, bases that are different from the bases adjacent to both sides of the point where the same base is repeated.

According to an embodiment of the present specification, the step (c) may be a step in which the processor performs a secondary conversion by adding dummy bases having two or more sequences.

The DNA encoding method according to the present specification may be implemented in the form of a computer program recorded on a computer-readable recording medium, the computer program being configured to execute each step of the DNA encoding method on a computer.

According to an embodiment of the present specification, a DNA encoding apparatus for solving the above-described problems may comprise: a processor configured to perform a primary conversion of binary data into a base sequence, identify a point at which the same base is repeated in the primarily converted base sequence, and perform a secondary conversion by adding dummy bases at the point where the same base is repeated.

According to an embodiment of the present specification, the processor may identify a point at which a predetermined number of identical bases are repeated.

According to an embodiment of the present specification, the processor may identify a point at which the same base is repeated based on a predefined number set for each base.

According to an embodiment of the present specification, the processor may generate a frequency table for repeated bases in the primarily converted base sequence, determine the number of repeated identical bases requiring dummy base addition using the generated frequency table, and identify the point where the same base is repeated based on the determined number.

According to an embodiment of the present specification, the processor may determine the number of repeated identical bases requiring dummy base addition based on an average frequency value.

According to an embodiment of the present specification, the processor may determine, for each base, the number of repeated identical bases requiring dummy base addition based on an average frequency value for each base.

According to an embodiment of the present specification, the processor may perform a secondary conversion by adding dummy bases having at least one predetermined sequence.

According to an embodiment of the present specification, the processor may perform a secondary conversion by adding dummy bases that are different from the bases adjacent to both sides of the point where the same base is repeated.

According to an embodiment of the present specification, the processor may perform a secondary conversion by adding dummy bases having two or more sequences.

The DNA encoding apparatus according to the present specification may be a component of a DNA storage system comprising: a DNA encoding apparatus; and a DNA synthesizing apparatus configured to synthesize DNA based on the base sequence output from the DNA encoding apparatus.

According to an embodiment of the present specification, a binary data scrambling method for solving the above-described problems may comprise: (a) temporarily converting, by the processor, binary data into a base sequence; (b) determining, by the processor, whether there exists a point in the temporarily converted base sequence where the same base is repeated equal to or more than a predetermined number of times (hereinafter referred to as ‘repeated identical base sequence); (c) when a repeated identical base sequence exists in step (b), a step in which the processor reverse-converts the temporarily converted base sequence into binary data, scrambles the reversed binary data, and then returns to the step (a); and (d) when the repeated identical base sequence does not exist in the step (b), a step in which the processor reverse-converts the temporarily converted base sequence into binary data and stores the reversed binary data as target binary data for base sequence conversion.

According to an embodiment of the present specification, in step (b), the processor may identify a point at which a predetermined number of identical bases are repeated.

According to an embodiment of the present specification, in step (b), the processor may identify a point at which the same base is repeated based on a predefined number set for each base.

According to an embodiment of the present specification, in step (c), the processor may scramble the binary data using a Linear Feedback Shift Register (LFSR) scheme.

According to an embodiment of the present specification, step (c) may comprise: (c-1) storing, by the processor, the number of times the binary data has been scrambled; and (c-2) when the number of scrambling operations reaches a preset maximum, proceeding to step (d).

According to an embodiment of the present specification, in step (a), the processor may store the temporarily converted base sequence, and in step (c), the processor may further store the number of repeated identical bases in the temporarily converted base sequence; (c-1) when the reversed binary data is not identical to the original binary data, scrambling the reversed binary data and returning to step (a); and (c-2) when the reversed binary data is identical to the original binary data, sending the temporarily converted base sequence having the smallest number of repeated identical bases to step (d).

The binary data scrambling method according to the present specification may be implemented in the form of a computer program recorded on a computer-readable recording medium, the computer program being configured to execute each step of the binary data scrambling method on a computer.

According to an embodiment of the present specification, a binary data scrambling apparatus for solving the above-described problems may comprise: a base sequence conversion unit configured to convert binary data into a base sequence or reverse convert a base sequence into binary data; a repeated base analysis unit configured to determine whether a repeated identical base sequence, which refers to a point in the temporarily converted base sequence from the base sequence conversion unit where the same base is repeated equal to or more than a predetermined number of times, exists (hereinafter referred to as ‘repeated identical base sequence’); a binary data scrambling unit configured to scramble and output the binary data; and a control unit configured to control the base sequence conversion unit, the repeated base analysis unit, and the binary data scrambling unit. When the repeated base analysis unit determines that a repeated identical base sequence exists, the control unit may control the base sequence conversion unit to reverse convert the temporarily converted base sequence into binary data, scramble the reversed binary data, and convert the scrambled binary data back into a base sequence. When the repeated base analysis unit determines that no repeated base sequence exists, the control unit may control the base sequence conversion unit to reverse convert the temporarily converted base sequence into binary data and store the reversed binary data as target binary data for base sequence conversion.

According to an embodiment of the present specification, the repeated base analysis unit may identify a point at which a predetermined number of identical bases are repeated.

According to an embodiment of the present specification, the repeated base analysis unit may identify a point at which the same base is repeated based on a predefined number set for each base.

According to an embodiment of the present specification, the binary data scrambling unit may scramble the binary data using a Linear Feedback Shift Register (LFSR) scheme.

According to an embodiment of the present specification, the control unit may store the number of times the binary data has been scrambled by the binary data scrambling unit, and when a preset maximum number of scrambling operations is reached, reverse convert the temporarily converted base sequence into binary data and store the reversed binary data as target binary data for base sequence conversion.

According to an embodiment of the present specification, the control unit may store each temporarily converted base sequence by the base sequence conversion unit, additionally store the number of repeated identical bases analyzed by the repeated base analysis unit in the temporarily converted base sequence, and when the reversed binary data is not identical to the original binary data, control the binary data scrambling unit to scramble the reversed binary data, and when the reversed binary data is identical to the original binary data, store the temporarily converted base sequence with the smallest number of repeated identical bases as the target binary data for base sequence conversion.

The binary data scrambling apparatus according to the present specification may be a component of a DNA storage system comprising: a binary data scrambling apparatus; and a DNA synthesizing apparatus configured to synthesize DNA by converting the binary data output from the binary data scrambling apparatus into a base sequence.

According to an embodiment of the present specification, a binary data restoration method for solving the above-described problems may comprise: (a) receiving and storing, by the processor, analyzed DNA base sequence information; (b) reverse converting, by the processor, the base sequence information into binary data; (c) separating, by the processor, scrambling count information included in the reversed binary data; and (d) restoring, by the processor, the binary data from which the scrambling count information was separated, based on the scrambling count information.

According to an embodiment of the present specification, a binary data restoration method for solving the above-described problems may comprise: (a) receiving and storing, by the processor, analyzed DNA base sequence information; (b) reverse converting, by the processor, the base sequence information into binary data; (c) reading, by the processor, scrambling count information from a storage unit; and (d) restoring, by the processor, the reversed binary data based on the scrambling count information.

The binary data restoration method according to the present specification may further comprise a step of removing an error correction code from the reversed binary data after step (b).

In step (d) of the binary data restoration method according to the present specification, the processor may restore the binary data using a Linear Feedback Shift Register (LFSR) scheme.

The binary data restoration method according to the present specification may be implemented in the form of a computer program recorded on a computer-readable recording medium, the computer program being configured to execute each step of the binary data restoration method on a computer According to an embodiment of the present specification, a binary data restoration apparatus for solving the above-described problems may comprise: a base sequence conversion unit configured to reverse convert analyzed DNA base sequence information into binary data; a binary data processing unit configured to separate scrambling count information included in the reversed binary data; and a binary data descrambling unit configured to restore the binary data from which the scrambling count information was separated based on the scrambling count information.

According to an embodiment of the present specification, a binary data restoration apparatus for solving the above-described problems may comprise: a base sequence conversion unit configured to reverse convert analyzed DNA base sequence information into binary data; a storage unit configured to store scrambling count information for each DNA; and a binary data descrambling unit configured to restore the reversed binary data based on the scrambling count information stored in the storage unit.

The base sequence conversion unit of the binary data restoration apparatus according to the present specification may remove an error correction code from the reversed binary data.

The binary data descrambling unit of the binary data restoration apparatus according to the present specification
may restore the binary data using a Linear Feedback Shift Register (LFSR) scheme.

The binary data restoration apparatus according to the present specification may be a component of a DNA storage system comprising: a binary data restoration apparatus; and a base sequence analysis unit configured to analyze DNA and output base sequence information.

According to the present specification, a DNA encoding method for solving the above-described problems may comprise: (a) a step in which the processor performs a primary conversion of binary data into base sequence information; and (b) a step in which the processor performs a secondary conversion into base sequence information to which dummy base information is added at every predetermined interval in the primarily converted base sequence information.

According to an embodiment of the present specification, the dummy base information added in step (b) may be base information different from the base information adjacent in the primarily converted base sequence information.

According to an embodiment of the present specification, the dummy base information added in step (b) may comprise at least two or more base information.

According to an embodiment of the present specification, the dummy base information added in step (b) may be such that the base information at both ends of the added dummy base information differs from the adjacent base information in the primarily converted base sequence information The DNA encoding method according to the present specification may further comprise: (c) performing a tertiary conversion, by the processor, into base sequence information to which protective dummy base information is added at both terminal ends of the secondarily converted base sequence information.

The DNA encoding method according to the present specification may be implemented in the form of a computer program recorded on a computer-readable recording medium, the computer program being configured to execute each step of the DNA encoding method on a computer.

According to an embodiment of the present specification, a DNA encoding apparatus for solving the above-described problems may comprise: a processor configured to perform a primary conversion of binary data into base sequence information, and perform a secondary conversion of the base sequence information into base sequence information to which dummy base information is added at predetermined intervals in the primarily converted base sequence information.

According to an embodiment of the present specification, the processor may add dummy base information that differs from adjacent base information in the primarily converted base sequence information.

According to an embodiment of the present specification, the processor may add dummy base information composed of at least two or more base information.

According to an embodiment of the present specification, the processor may add dummy base information such that the bases located at both ends of the dummy base information are different from the adjacent bases in the primarily converted base sequence information.

According to an embodiment of the present specification, the processor may further perform a tertiary conversion into base sequence information to which protective dummy base information is added at both terminal ends of the secondarily converted base sequence information.

The DNA encoding apparatus according to the present specification may be a component of a DNA storage system comprising: a DNA encoding apparatus; and a DNA synthesizing apparatus configured to synthesize DNA based on the base sequence output from the DNA encoding apparatus.

Other specific details of the present invention are included in the detailed description and drawings below.

### Effects of the Invention

According to the present specification, it is possible to reduce the possibility of errors during DNA synthesis and restoration. In addition, the structural stability of the synthesized DNA can be enhanced.

The effects of the present invention are not limited to those mentioned above, and other effects not mentioned will be clearly understood by those skilled in the art from the following description.

### BRIEF DESCRIPTION OF THE DRAWINGS

FIG. 1 is a conceptual diagram of information storage using a DNA base sequence.

FIG. 2 is a reference diagram of a DNA information storage system according to the present specification.

FIG. 3 is a flowchart of a DNA encoding method according to the present specification.

FIG. 4 is an example diagram of a DNA encoding method according to the present specification.

FIG. 5 is an example of dummy bases that may be added depending on the point at which the same base is repeated and the adjacent base that follows.

FIG. 6 is a schematic configuration diagram of a binary data scrambling apparatus according to the present specification.

FIG. 7 is a flowchart of a binary data scrambling method according to an embodiment of the present specification.

FIG. 8 is a reference diagram of binary data conversion into a base sequence.

FIG. 9 is a reference diagram to aid in understanding a linear feedback shift register.

FIG. 10 is a flowchart of a binary data scrambling method according to another embodiment of the present specification.

FIG. 11 is a flowchart of a binary data scrambling method according to still another embodiment of the present specification.

FIG. 12 is a block diagram schematically illustrating the configuration of a binary data restoration apparatus according to an embodiment of the present specification.

FIG. 13 is a block diagram schematically illustrating the configuration of a binary data restoration apparatus according to another embodiment of the present specification.

FIG. 14 is a flowchart of a binary data restoration method according to an embodiment of the present specification.

FIG. 15 is a flowchart of a binary data restoration method according to another embodiment of the present specification.

FIG. 16 is a flowchart of a DNA encoding method according to the present specification.

FIG. 17 is an example diagram of a DNA encoding method according to an embodiment of the present specification.

FIG. 18 is a table of types of dummy bases that may be added depending on adjacent bases.

FIG. 19 is an example diagram of a DNA encoding method according to another embodiment of the present specification.

FIG. 20 is an example diagram of a DNA encoding method according to still another embodiment of the present specification.

### MODES FOR CARRYING OUT THE INVENTION

The advantages and features of the invention disclosed in the present specification, as well as methods for achieving them, will become apparent with reference to the embodiments described in detail below in conjunction with the accompanying drawings.

However, the present specification is not limited to the embodiments disclosed below and may be implemented in various other forms. These embodiments are provided merely to ensure the completeness of the disclosure and to fully convey the scope of the present specification to those of ordinary skill in the art (hereinafter referred to as “skilled artisans”). The scope of rights of the present specification is defined solely by the scope of the claims.

The terminology used in this specification is intended to describe particular embodiments and is not intended to limit the scope of the claims. Unless specifically stated otherwise, the singular forms include the plural forms as well. The terms “comprises” and/or “comprising” used in this specification do not exclude the presence or addition of one or more other components not explicitly mentioned.

Throughout the specification, identical reference numerals refer to the same components, and the expression “and/or” includes each and every combination of the components referred to. Although terms such as “first” and “second” may be used to describe various components, such components are not limited by these terms. These terms are used only to distinguish one component from another. Therefore, a “first” component mentioned below may also be referred to as a “second” component within the technical scope of the present invention.

Unless otherwise defined, all terms (including technical and scientific terms) used in this specification are to be interpreted as having meanings commonly understood by those skilled in the art to which this specification pertains. Terms generally defined in commonly used dictionaries should be interpreted as having meanings consistent with those in the context of the related art and should not be interpreted in an idealized or overly formal manner unless explicitly defined otherwise herein.

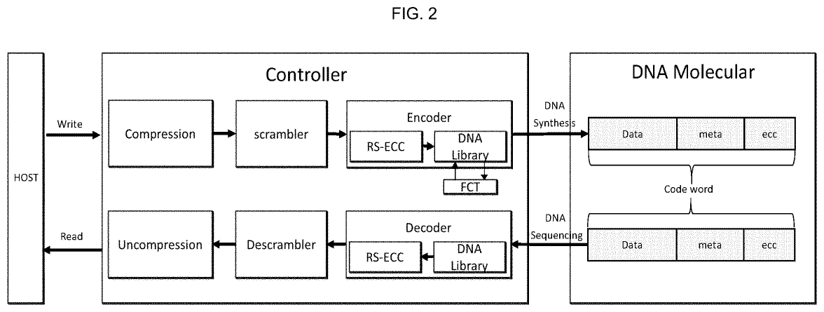

FIG. 2 is a reference diagram of a DNA-based data storage system according to the present specification.

Referring to FIG. 2, the DNA-based data storage system may be broadly divided into a controller and a DNA molecular unit. The controller receives a write request from a host and may compress the binary data. The compressed data may be scrambled. The purpose of scrambling is to prevent repetition of identical nucleotide sequences when the binary data is later converted into nucleotide sequences. After the scrambling process is completed, error correction codes (ECC) may be added. Then, the binary data may be converted into corresponding nucleotide sequence information (DNA library). The DNA molecular unit may synthesize actual DNA molecules based on the nucleotide sequence-converted data.

When a read request is made to retrieve the information stored as DNA molecules, the DNA molecular unit performs sequencing to analyze the nucleotide sequences of the DNA molecules. The controller converts the analyzed nucleotide sequences back into binary data and uses error correction codes (ECC) to correct any errors. The corrected binary data is then descrambled and decompressed to restore the original binary data (information).

In the technical field to which this specification pertains, it is known that synthesis errors may occur when identical nucleotides are repeatedly synthesized during the DNA synthesis process. According to Poon and MacGregor (1988) Biopolymers 45:427-434, when guanine (G) is repeatedly synthesized four or more times, there is a problem of aggregation into a guanine tetraplex structure. Although this literature addresses the problem with guanine (G), it does not exclude the possibility that similar issues may arise with adenine (A), thymine (T), or cytosine (C) as well. Therefore, the applicant has recognized the need for a method to reduce the likelihood of synthesis errors in DNA-based information storage systems.

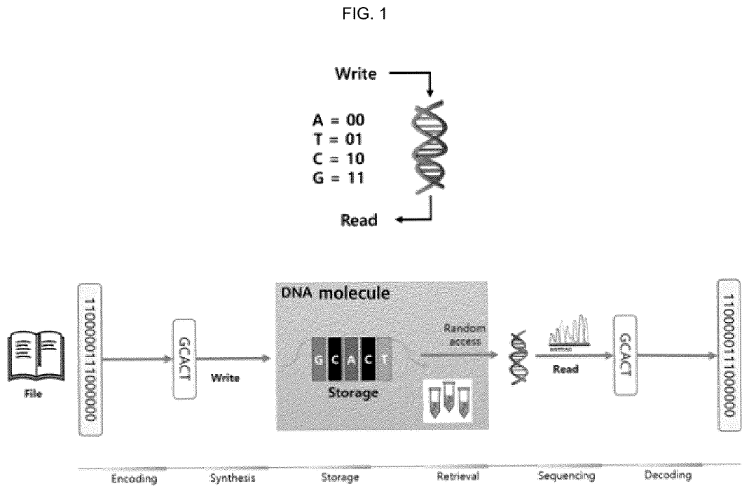

Hereinafter, a DNA encoding method according to the present specification will be described with reference to the accompanying drawings. The DNA encoding method according to the present specification corresponds to the step of converting binary data into nucleotide sequences, as shown in FIG. 1. DNA synthesized in the future may be based on the encoded nucleotide sequences. Accordingly, DNA encoding refers to the step of determining the arrangement order of nucleotides. In this specification, the reading direction of nucleotide sequences is defined from the 5 ′ end to the 3′ end. Repetition of nucleotide sequences refers to consecutive placement of identical nucleotides. Therefore, a repeated nucleotide sequence refers to a position where the same nucleotide is consecutively placed in a forward direction a predetermined number of times.

Meanwhile, the DNA encoding method according to the present specification may be implemented in the form of a computer program recorded on a computer-readable recording medium, the program being configured to execute each of the steps described below. When the DNA encoding method according to the present specification is implemented as a computer program, each step may be executed by a processor.

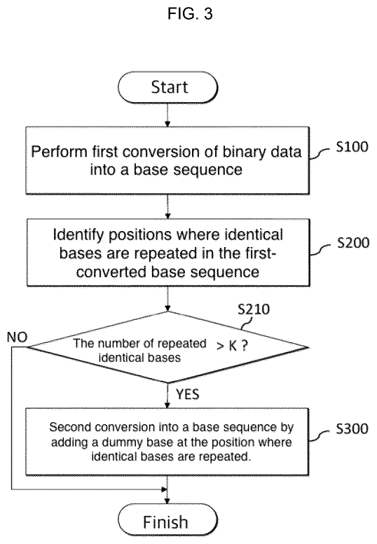

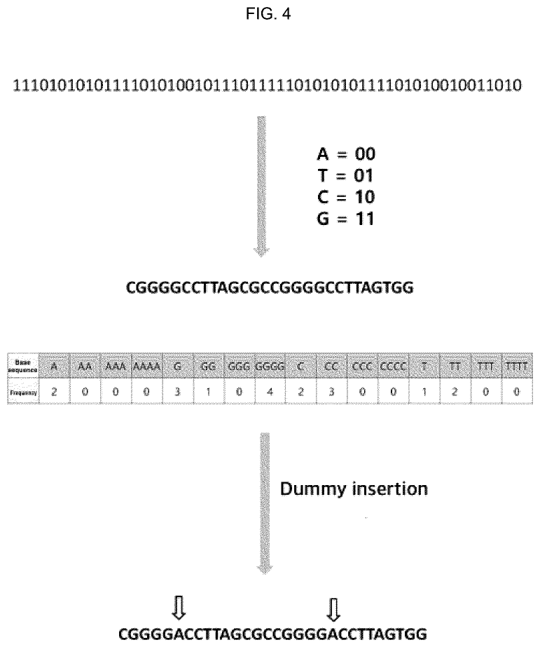

FIG. 3 is a flowchart of the DNA encoding method according to the present specification. FIG. 4 is an exemplary diagram of the DNA encoding method according to the present specification.

Referring to FIG. 3, the processor may first perform a first conversion of binary data into a nucleotide sequence in step S100. In the next step S200, the processor may identify positions in the first-converted nucleotide sequence where identical nucleotides are repeated. In the next step S300, the processor may perform a second conversion into a nucleotide sequence in which dummy nucleotides are inserted at the identified repeated positions of the same nucleotides.

First, let us look more closely at step S100, in which binary data is first converted into a nucleotide sequence. According to one example, the processor may map each 2 bits of binary data to a nucleotide as follows: A (adenine)=00, T (thymine) =01, G (guanine)=10, and C (cytosine)=11, thereby performing the first conversion into a nucleotide sequence. In the example shown in FIG. 4, each 2-bit binary data is mapped 1:1 to a nucleotide. However, the DNA encoding method according to the present specification is not limited to the example shown. One nucleotide may correspond to 1 bit, 2 bits, 3 bits, or 4 bits, and various mapping methods may be used between binary data and nucleotide sequences.

Let us now examine in more detail the process in step S200 of identifying positions in the first-converted nucleotide sequence where identical nucleotides are repeated. As previously explained, when a specific nucleotide is repeatedly (continuously) placed, there is a high likelihood of synthesis errors occurring during the DNA synthesis process. In other words, step S200 corresponds to a process of detecting positions where synthesis defects are likely to occur.

According to one embodiment of the present specification, the processor may identify positions where a predetermined number of identical nucleotides are consecutively repeated. The predetermined number may vary, for example, from 4 to 70 or more. A uniform criterion may be applied to all four nucleotides, or different criteria may be applied to each nucleotide individually. Accordingly, the processor may identify positions of repeated identical nucleotides based on a preset threshold specific to each nucleotide. For example, A may have a threshold of 5, T a threshold of 6, G a threshold of 7, and C a threshold of 5. In this way, the threshold count may vary between different nucleotides, and some nucleotides may share the same threshold value.

According to another embodiment of the present specification, the threshold may be determined based on the number of repetitions of identical nucleotides in the first-converted nucleotide sequence. To achieve this, the processor may generate a frequency count table (see FIG. 4 for an example) of repeated nucleotides in the first-converted nucleotide sequence. Then, the processor may determine the number of repeated identical nucleotides at which dummy nucleotides need to be inserted, based on the generated frequency table. The processor may subsequently identify the positions of repeated identical nucleotides in the first-converted nucleotide sequence, according to the determined repetition count. It is noted that the frequency count table may be included in the DNA library component within the controller of the DNA information storage system according to the present specification, which is responsible for converting binary information into nucleotide sequence information.

There are various methods for determining the required repetition count of identical nucleotides at which dummy nucleotides should be inserted using the frequency count table. In one example, the processor may determine the average frequency and use it as the repetition threshold for inserting dummy nucleotides. In the example shown in FIG. 4, the average frequency is 1.125. By applying a ceiling operation, it may be determined that a dummy nucleotide should be inserted at every point where two identical nucleotides are consecutively repeated. Furthermore, the processor may calculate the average frequency for each individual nucleotide and determine the repetition threshold for dummy nucleotide insertion accordingly. In the example shown in FIG. 4, the average for A is 0.5, for G is 2, for C is 1.25, and for T is 0.75. In this case, thresholds below 2, which do not indicate meaningful repetition, may be excluded. Therefore, dummy nucleotides may not be inserted when A, C, or T are repeated, while insertion may be performed only when G is repeated two or more times.

In step S210, the processor may determine whether the number of repeated identical nucleotides exceeds a predetermined threshold based on various embodiments described above. If the number of repeated identical nucleotides does not exceed the threshold (i.e., “NO” in step S210), then insertion of a dummy nucleotide is not necessary. On the other hand, if the number exceeds the threshold (i.e., “YES” in step S210), the corresponding position is determined to require dummy nucleotide insertion, and the process proceeds to step S300.

In step S300, the process of performing a second conversion by inserting dummy nucleotides at positions where identical nucleotides are repeated will now be described in more detail. According to one embodiment of the present specification, the processor may perform the second conversion by inserting at least one predefined dummy nucleotide sequence. In the example shown in FIG. 4, when four guanine (G) nucleotides are consecutively repeated, adenine (A) is inserted as a dummy nucleotide. However, the DNA encoding method according to the present specification is not limited to the example shown in FIG. 4. Although the dummy nucleotide in the example is a single ‘A’, other nucleotides such as G, T, or C may also be used as dummy nucleotides. Furthermore, the number of dummy nucleotides may be more than one. For example, combinations such as ‘AA’, ‘GG’, ‘TT’, ‘CC’, ‘AG’, ‘GT’, ‘TC’, and ‘CA’ may be used. A variety of combinations are possible.

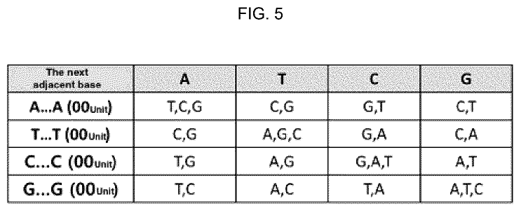

Meanwhile, as previously explained, the reason for inserting dummy nucleotides is to prevent the repeated placement of identical nucleotides. Preferably, the processor may perform the second conversion by adding a dummy nucleotide that is different from the nucleotides adjacent to both sides of the repeated identical nucleotide region. FIG. 5 illustrates examples of dummy nucleotides that may be inserted depending on the repeated nucleotide and the adjacent nucleotides. In the example shown in FIG. 5, for the sake of simplicity and clarity of explanation, a case where the dummy nucleotide consists of a single nucleotide is depicted. However, as described earlier, the processor may also perform the second conversion by inserting a dummy nucleotide sequence comprising two or more nucleotides. In such cases, dummy nucleotides composed of sequences different from the adjacent nucleotides on both sides of the repeated region may also be inserted.

In the present specification, the term “binary data” refers to data composed of 0s and 1s. The term “nucleotide sequence” refers to information composed of A (adenine), T (thymine), G (guanine), and C (cytosine). The reading direction of the nucleotide sequence is defined as the forward direction from the 5′ end to the 3′ end. Repetition of a nucleotide sequence refers to the continuous placement of identical nucleotides. Therefore, a repeated nucleotide sequence position refers to a position where the same nucleotide is repeatedly arranged in the forward direction a predetermined number of times.

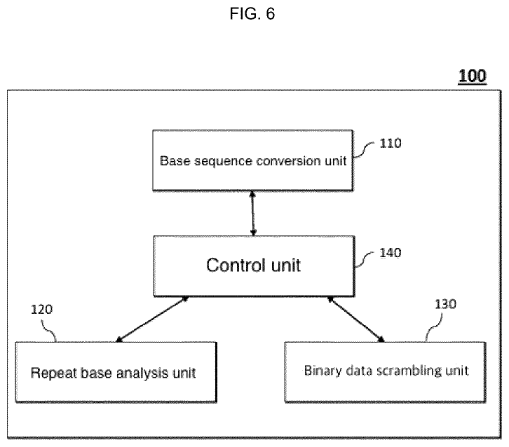

FIG. 6 is a schematic block diagram of a binary data scrambling apparatus according to the present specification.

Referring to FIG. 6, the binary data scrambling apparatus 100 may include a nucleotide sequence conversion unit 110, a repeated nucleotide analysis unit 120, a binary data scrambling unit 130, and a control unit 140. The nucleotide sequence conversion unit 110 may convert binary data into a nucleotide sequence or vice versa. The repeated nucleotide analysis unit 120 may determine whether a region (hereinafter referred to as a “repeated nucleotide sequence”) exists in the temporarily converted nucleotide sequence in which the same nucleotide is repeated equal to or more than a predetermined number of times. The binary data scrambling unit 130 may scramble and output the binary data. The control unit 140 may control the nucleotide sequence conversion unit 110, the repeated nucleotide analysis unit 120, and the binary data scrambling unit 130. When the repeated nucleotide analysis unit 120 determines that a repeated nucleotide sequence exists, the control unit 140 may reconvert the temporarily converted nucleotide sequence back into binary data, scramble the reconverted binary data, and then convert the scrambled binary data back into a nucleotide sequence. In contrast, when the repeated nucleotide analysis unit 120 determines that no repeated nucleotide sequence is present, the control unit 140 may reconvert the temporarily converted nucleotide sequence into binary data and store the reconverted binary data as the binary data to be converted into a nucleotide sequence.

Meanwhile, the nucleotide sequence conversion unit 110, the repeated nucleotide analysis unit 120, the binary data scrambling unit 130, and the control unit 140 may include, for example, a processor, an ASIC (application-specific integrated circuit), other chipsets, logic circuits, registers, communication modems, data processing devices, or other components known in the technical field to which the present invention pertains, in order to execute the binary data scrambling method described below. In addition, when the control logic described below is implemented in software, the nucleotide sequence conversion unit 110, the repeated nucleotide analysis unit 120, the binary data scrambling unit 130, and the control unit 140 may be implemented as a set of program modules. The program modules may be stored in a memory device and executed by a processor. Accordingly, the binary data scrambling method according to the present specification may be implemented in the form of a computer program stored on a computer-readable recording medium, the program being configured to perform each of the steps described below. The binary data scrambling method according to the present specification will be described hereinafter on the assumption that it is executed by a processor.

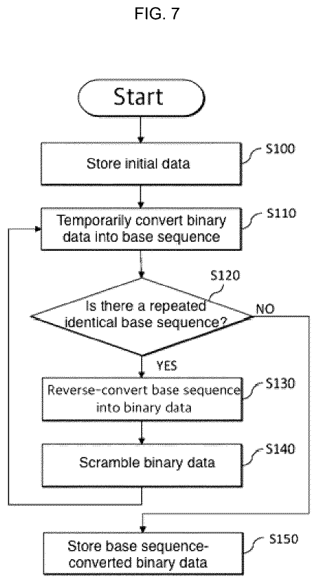

FIG. 7 is a flowchart of a binary data scrambling method according to an embodiment of the present specification.

Referring to FIG. 7, in step S100, the processor may receive and store initial data. In the present specification, the term “initial data” refers to original binary data that has not been scrambled. In step S110, the processor may temporarily convert the initial data (binary data) into a base sequence. The term “temporary base sequence” used for the sequence converted in step S110 is to distinguish it from “target binary data for base sequence conversion,” which will be described later. The temporary base sequence may not necessarily be the base sequence that will be synthesized into actual DNA. In contrast, the target binary data for base sequence conversion corresponds to the base sequence that will actually be synthesized into DNA.

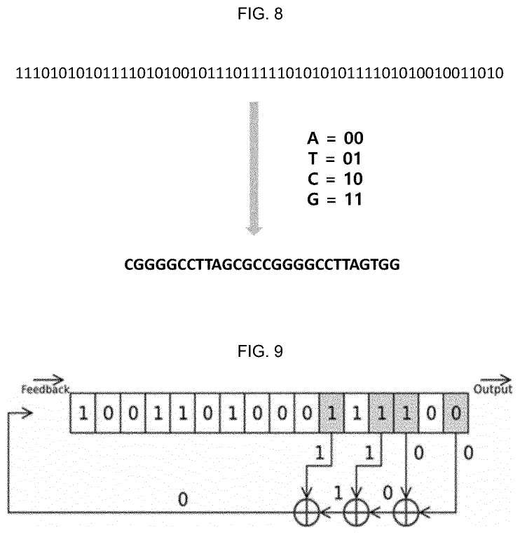

FIG. 8 is a reference diagram for base sequence conversion of binary data.

Referring to FIG. 8, the processor may perform a primary conversion of binary data into a base sequence by mapping each 2-bit segment to a corresponding base: A (adenine)=00, T (thymine)=01, G (guanine)=10, and C (cytosine)=11. Although the example illustrated in FIG. 8 shows a one-to-one mapping between 2-bit data and bases, the binary data scrambling method according to the present specification is not limited to the illustrated example. One base may correspond to 1, 2, 3, or 4 bits, and various mapping methods between binary data and base sequences may be used.

Referring back to FIG. 7, in step S120, the processor may determine whether there exists a point in the temporarily converted base sequence at which the same base is repeated equal to or more than a predetermined number of times (hereinafter referred to as ‘repeated identical base sequence’). As described above, a repeated (consecutive) arrangement of specific bases can increase the likelihood of errors during the synthesis process. Accordingly, step S120 is a process of identifying a point in the base sequence that is likely to cause a defect during the DNA synthesis process.

According to an embodiment of the present specification, the processor may identify a point at which a predetermined number of identical bases are repeated. For example, the predetermined number may be set to various values such as 4 to 70. Meanwhile, the same threshold may be applied to all four bases, but different thresholds may also be applied individually to each base. Accordingly, the processor may identify a point at which the same base is repeated, based on a predefined number set for each base. For example, A may have a threshold of 5, T may have a threshold of 6, G may have a threshold of 7, and C may have a threshold of 5. In this manner, different bases may have different threshold values, and some bases may share the same threshold.

If a repeated identical base sequence exists, meaning that the number of repeated identical bases is equal to or more than the predefined threshold ('YES′ in step S120), the process proceeds to step S130. In step S130, the processor may reverse-convert the temporarily converted base sequence into binary data. In step S140, the processor may scramble the reversed binary data and then proceed to step S110. That is, if the number of repeated identical bases in the converted base sequence is equal to or more than a predetermined threshold, steps S110 to S140 may be repeatedly executed.

On the other hand, if no repeated identical base sequence exists, meaning that the number of repeated identical bases is less than a predetermined threshold (‘NO’ in step S120), the process proceeds to step S150. In step S150, the processor may reverse-convert the temporarily converted base sequence into binary data and store the reversed binary data as target binary data for base sequence conversion. The reason for reverse-converting the base sequence into binary data is that the binary data may need to be further processed by the encoder illustrated in FIG. 2.

The method of scrambling the binary data in step S140 may vary. According to an embodiment of the present specification, the processor may scramble the binary data using a Linear Feedback Shift Register (LFSR) scheme. An LFSR is a type of shift register in which the input value is calculated as a linear function of previous state values. The most commonly used linear function is the exclusive OR (XOR). The initial bit value of the LFSR is called a seed. LFSRs are used in various fields such as pseudorandom number generation, pseudorandom noise (PRN), fast digital counters, and whitening sequences. In the present specification, the LFSR, which is conventionally used for generating pseudorandom numbers and the like, is utilized as an element for addressing the issue of repeated identical bases.

FIG. 9 is a reference diagram to aid in understanding a Linear Feedback Shift Register.

The tap sequence of an LFSR can be expressed as a polynomial congruence equation, where the coefficients of the polynomial must be either 1 or 0. This is referred to as the feedback polynomial or characteristic polynomial. For example, if the taps are the 16th, 14th, 13th, and 11th bits, the LFSR polynomial is as follows:
X¹¹+x¹³+X¹⁴+X¹⁶+1

A coefficient of 1 does not necessarily correspond to a tap. The length of the LFSR may be designed according to the length of the DNA to be synthesized. For example, when the length of the DNA strand to be synthesized is 150 bases, the length of the LFSR is 2n-1. Accordingly, if n is 8, the LFSR has a length of 255, and an 8th-order polynomial is sufficient to process a DNA strand of 150 bases. If the length of the DNA strand to be synthesized exceeds 150 bases, then with n set to 10, the LFSR can generate 1023 values. Accordingly, the number of polynomials used in the LFSR may be increased in proportion to the length of the DNA strand. In an LFSR, depending on the position of the XOR gates, it may be implemented as an ‘external LFSR’ or an ‘internal LFSR’, and various types such as a ‘Galois LFSR’, which are well known to those skilled in the art, may also be applied. Further detailed explanation of the LFSR is omitted as it is known to those skilled in the art.

Meanwhile, the operation of the LFSR is deterministic. Thus, the sequence of values generated by the LFSR is determined by its previous state. Since the register can have only a finite number of states, the sequence may eventually repeat with a specific period. Of course, if an appropriate linear function is selected, it is possible to generate a sequence with a long period that appears random. However, if the values output from the LFSR are continuously re-input into the LFSR, there is a possibility that the sequence may be repeated. That is, in some cases, if steps S110 to S140 are repeatedly executed, the original data may be output again. Therefore, it is necessary to prevent infinite repetition of steps S110 to S140.

One method for preventing repeated execution of the LFSR is to predefine the number of executions.

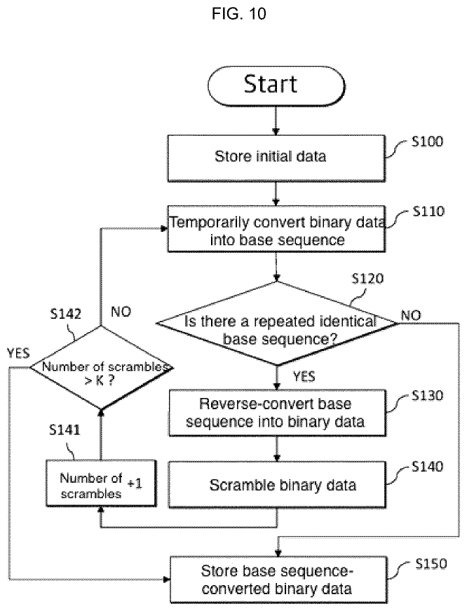

FIG. 10 is a flowchart of a binary data scrambling method according to another embodiment of the present specification.

Referring to FIG. 10, it can be seen that steps S100 to S150 remain the same, and steps S141 and S142 are additionally included. According to another embodiment of the present specification, after scrambling the binary data in step S140, the processor may proceed to step S141. In step S141, the processor may store the number of times the binary data is scrambled whenever the scrambling is performed. And, in step S142, the processor determines whether the number of scrambling iterations has exceeded a preset maximum value (K). If the number of iterations is less than K (‘NO’ in step S142), the process may proceed to step S110. That is, steps S110 to S142 may be repeatedly executed until the number of times the data has been scrambled reaches the maximum number of scrambles(K). If the number of iterations exceeds K (‘YES’ in step S142), the processor proceeds to step S150. That is, without performing additional scrambling, the finally scrambled binary data is stored as the base sequence-converted binary data.

Another method for preventing repeated execution of the LSFR is to identify the binary data, among the converted binary data, that has the least number of repeated occurrences of base sequences.

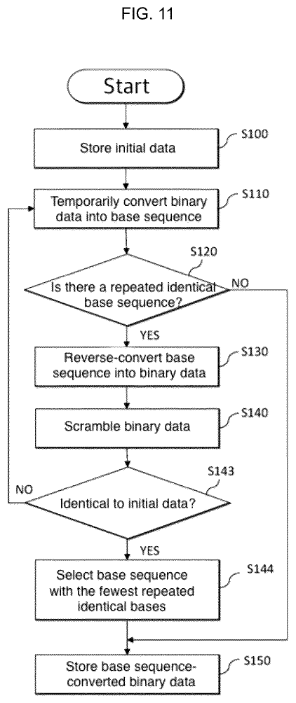

FIG. 11 is a flowchart of a binary data scrambling method according to still another embodiment of the present specification.

Referring to FIG. 11, it can be seen that steps S143 and S144 are additionally included. In step S110, the processor may store each temporarily converted base sequence. That is, each time step S110 is executed after the binary data is scrambled, the converted temporary data may be stored. In step S120, the processor may additionally store the number of repeated identical bases in each temporarily converted base sequence. That is, information indicating the actual number of identically repeated bases in the temporarily converted base sequence may additionally be stored. Steps S130 and S140 are the same as previously described. In step S143, following step S140, the processor may determine whether the reverse-converted binary data is identical to the original binary data, the initial data.

If the reverse-converted binary data is not identical to the original binary data (‘NO’ in step S143), the process may proceed to step S110. Subsequently, the processor may be configured to repeatedly perform steps S110 to S143. The repeated execution of steps S110 to S143 may be performed until the number of consecutively repeated identical bases in step S120 is equal to or less than a reference value, or until the scrambled binary data is identical to the original binary data.

On the other hand, if the reverse-converted binary data is identical to the original binary data (YES in step S143), the process may proceed to step S144. In step S144, the processor may select the base sequence having the smallest number of identically repeated bases among the temporarily converted base sequences. In step S150, the processor may reverse-convert the selected base sequence back into binary data, and may store the reverse-converted binary data as the binary data to be subjected to base sequence conversion.

In one embodiment, the processor may add scrambling count information to the binary data for base sequence conversion. For example, if no repeated identical base sequence exists in the initially converted base sequence, the scrambling count may be 0. Also, if steps S120 to S140 are repeatedly executed twice until no identical base repeat sequence remains, the scrambling count information may be ‘2’. As such, information on how many times the binary data for base sequence conversion has undergone scrambling may be added to the binary data.

In another embodiment, the processor may store the scrambling count information of the binary data for base sequence conversion in a separate storage device. While the foregoing embodiment illustrates an example where the scrambling count information is converted into a base sequence and recorded in the DNA itself, the present alternative embodiment illustrates an example in which the scrambling count information is stored in a separate storage device.

The following describes a method of reading and restoring information stored in the DNA storage system. According to the present specification, the DNA storage system is a state in which the binary data, finalized as the target for base sequence conversion through the previously described binary data scrambling method and apparatus, is synthesized into an actual DNA molecule based on the base sequence. Accordingly, when a request to read the information stored in the DNA molecule is generated, a process of descrambling and restoring the original binary data is required. The role of the binary data restoration method and apparatus according to the present specification is to perform such descrambling and restoration of the original binary data.

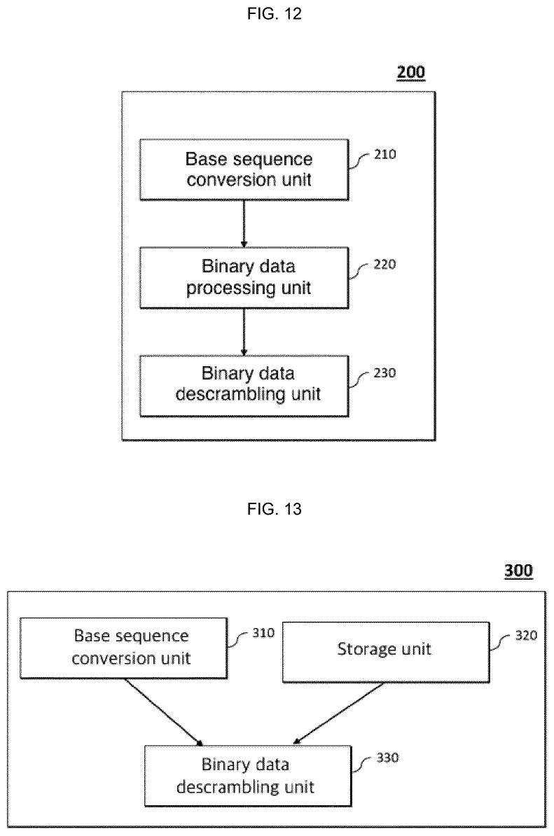

Meanwhile, the binary data restoration apparatus according to the present specification may constitute one component of a DNA storage system. The DNA storage system may include a base sequence analysis unit configured to analyze DNA and output base sequence information. Therefore, the binary data restoration apparatus and method according to the present specification are premised on having received information resulting from the analysis of the base sequence of an actual DNA molecule. Subsequently, an important aspect of the restoration process is that restoration must be performed based on the scrambling count information, which indicates how many times the DNA molecule has undergone scrambling. As described above, the scrambling count information may be stored within the DNA molecule itself, or may be stored in a separate storage device FIG. 12 is a block diagram schematically illustrating the configuration of a binary data restoration apparatus according to an embodiment of the present specification.

Referring to FIG. 12, the binary data restoration apparatus (200) may comprise a base sequence conversion unit (210), a binary data processing unit (220), and a binary data descrambling unit (230). The base sequence conversion unit (210) may reverse-convert the analyzed DNA base sequence into binary data. The base sequence conversion unit (210) may be identical or similar to the base sequence conversion unit (110) of the binary data scrambling apparatus (100) described above. The binary data processing unit (220) may extract the embedded scrambling count information from the reversed binary data. The binary data restoration apparatus (200) according to an embodiment of the present specification corresponds to an embodiment in which the scrambling count information is included in the DNA molecule. Accordingly, the reverse-converted binary data includes the scrambling count information, and the original binary data can be restored through descrambling only after the scrambling count information has been removed. The binary data descrambling unit (230) may restore the binary data, from which the scrambling count information has been separated, based on the scrambling count information.

FIG. 13 is a block diagram schematically illustrating the configuration of a binary data restoration apparatus according to another embodiment of the present specification.

Referring to FIG. 13, a binary data restoration apparatus (300) according to another embodiment of the present specification may include a base sequence conversion unit (310), a storage unit (320), and a binary data descrambling unit (330). The base sequence conversion unit (310) may reverse-convert the analyzed DNA base sequence information into binary data. The base sequence conversion unit (310) may be identical or similar to the base sequence conversion unit (110) of the binary data scrambling apparatus (100) described above. The storage unit (320) may store scrambling count information for each DNA molecule. The binary data restoration apparatus(300) according to another embodiment of the present specification corresponds to an embodiment in which the scrambling count information is not included in the DNA molecule. Although the storage unit(320) is illustrated as being included within the binary data restoration apparatus(300) in the present specification, the storage unit(320) may alternatively be located outside the binary data restoration apparatus(300). The binary data descrambling unit(330) may restore the reverse-converted binary data based on the scrambling count information stored in the storage unit(320).

Meanwhile, the base sequence conversion unit(210, 310) may remove an error correction code from the reverse-converted binary data. When storing binary data, an error correction code may be added and then converted into a base sequence. Accordingly, it is also necessary to remove the error correction code from the reverse-converted binary data.

The binary data descrambling units(230, 330) may restore the binary data using the LFSR method. This corresponds to the case where the binary data has been scrambled using a Linear Feedback Shift Register (LFSR) method. The Linear Feedback Shift Register (LFSR) allows the original data to be restored when executed in reverse, similar to the scrambling process. As the LFSR is known to those skilled in the art, a detailed description of its algorithm will be omitted.

Meanwhile, the base sequence conversion units(210, 310), the binary data processing unit(220), the binary data descrambling units(230,330), and the storage unit(320) may include a processor, an application-specific integrated circuit (ASIC), another chipset, logic circuits, registers, communication modems, data processing devices, or the like known in the art, in order to execute the binary data restoration method to be described below. In addition, when the control logic described below is implemented in software, the base sequence conversion units(210, 310), the binary data processing unit(220), and the binary data descrambling units(230, 330) may be implemented as a set of program modules. In this case, the program modules may be stored in a memory device and executed by a processor. Accordingly, the binary data restoration method according to the present specification may be implemented in the form of a computer program recorded on a computer-readable recording medium, the computer program being configured to perform each of the steps to be described below. Hereinafter, the binary data restoration method according to the present specification will be described on the premise that it is executed by a processor.

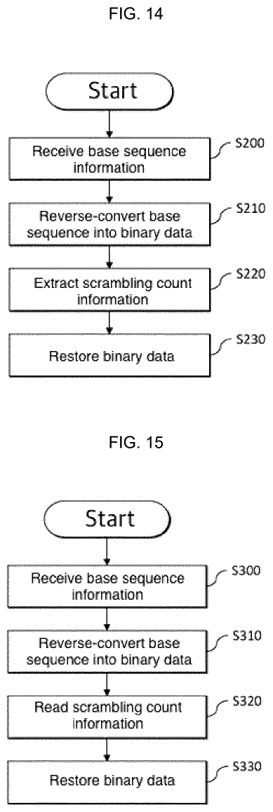

FIG. 14 is a flowchart illustrating a binary data restoration method according to an embodiment of the present specification.

The binary data restoration method according to an embodiment of the present specification corresponds to an embodiment in which the scrambling count information is included in the DNA molecule, as in the embodiment shown in FIG. 12.

Referring to FIG. 14, in step S210, the processor may receive and store analyzed DNA base sequence information. In the next step S210, the processor may reverse-convert the base sequence information into binary data. In step S220, the processor may separate the scrambling count information included in the reverse-converted binary data. Then, in step S230, the processor may restore the binary data, from which the scrambling count information has been separated, based on the scrambling count information.

FIG. 15 is a flowchart illustrating a binary data restoration method according to another embodiment of the present specification.

The binary data restoration method according to another embodiment of the present specification corresponds to an embodiment in which the scrambling count information is not included in the DNA molecule, as in the embodiment shown in FIG. 13.

Referring to FIG. 15, in step S310, the processor may receive and store analyzed DNA base sequence information. In the next step S310, the processor may reverse-convert the base sequence information into binary data. In step S320, the processor may read the scrambling count information from the storage unit. Then, in step S330, the processor may restore the binary data, from which the scrambling count information has been separated, based on the scrambling count information.

Meanwhile, after step S210 or S310, the processor may remove the error correction code from the reverse-converted binary data.

In addition, in step S230 or S330, the processor may restore the binary data using a Linear Feedback Shift Register (LFSR) method.

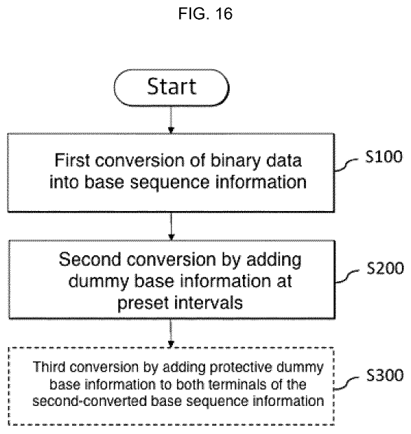

FIG. 16 is a flowchart of a DNA encoding method according to the present specification.

Referring to FIG. 16, in step S100, the processor may perform a first conversion of binary data into base sequence information. In the next step S200, the processor may perform a second conversion by inserting dummy base information into the first-converted base sequence information at predetermined intervals. For ease of understanding, the DNA encoding method according to the present specification will be described with examples of binary data and base sequence information.

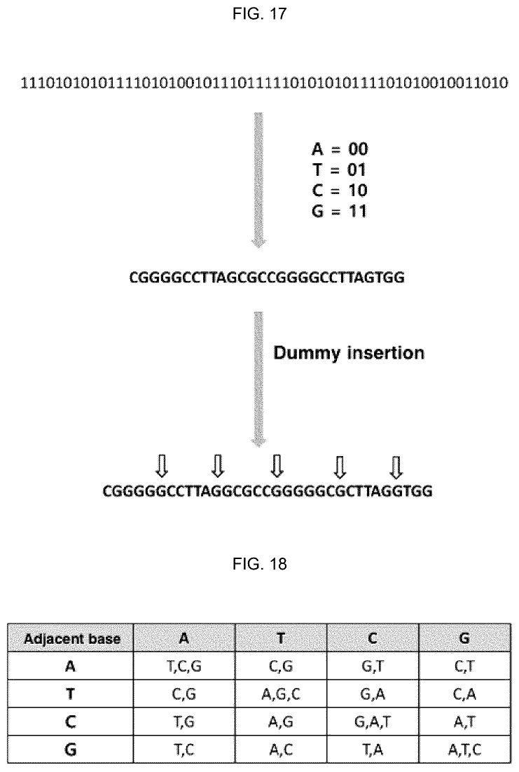

FIG. 17 is an exemplary diagram illustrating a DNA encoding method according to an embodiment of the present specification.

Referring to FIG. 17, the process of performing a first conversion of binary data into base sequence information in step S100 will be described in more detail. According to one example, the processor may perform the first conversion by mapping every2 bits of data to a base sequence information, such that A (adenine)=00, T (thymine)=01, G (guanine)=10, and C (cytosine)=11. While the example shown in FIG. 17 illustrates a 1:1 mapping between 2-bit data and bases, the DNA encoding method according to the present specification is not limited to this example. A single base may correspond to 1 bit, 2 bits, 3 bits, or 4 bits, and various methods may be used to match binary data with base information. The base sequence information converted in this manner is referred to as ‘first-converted base sequence information’.

Next, referring to FIG. 17, the process of adding dummy bases in step S200 will be described in more detail. In the example illustrated in FIG. 17, it can be seen that a dummy base “G (guanine)” is inserted every five base sequences. The interval at which the dummy base is inserted may be set in various ways. The shorter the interval, the greater the number of dummy bases inserted, which may reduce the risk of damage to actual data caused by ultraviolet radiation, thereby enhancing structural stability. However, a shorter interval also results in a reduced proportion of actual information within the DNA molecule. Therefore, the interval for inserting dummy bases may be appropriately determined in consideration of the storage environment and duration for the synthesized DNA molecules.

Meanwhile, according to one embodiment of the present specification, the dummy base information added in step S200 may be a base different from adjacent bases in the first-converted base sequence information. It is known that repeated synthesis of identical bases during DNA molecule synthesis may cause a possibility of defects. According to Poon and MacGregor (198) Biopolymers 45:427-434, continuous synthesis of four or more G(guanine) bases may lead to aggregation in the form of a guanine tetraplex. Although the above academic reference discusses issues related to G (guanine), it does not exclude the possibility that similar or identical problems may also occur with A (adenine), T (thymine), or C (cytosine). In the example shown in FIG. 17, it can be seen that the addition of the first, third, and fourth dummy bases from the left has resulted in a sequence of more than four consecutive G bases. Accordingly, in order to reduce the likelihood of synthesis defects, the dummy base may be varied depending on the adjacent base information. FIG. 18 is a table showing examples of dummy bases that may be added based on the adjacent bases.

Furthermore, although the example shown in FIG. 17 illustrates a dummy base consisting of a single base, according to one embodiment of the present specification, the added dummy base information may consist of at least two or more bases.

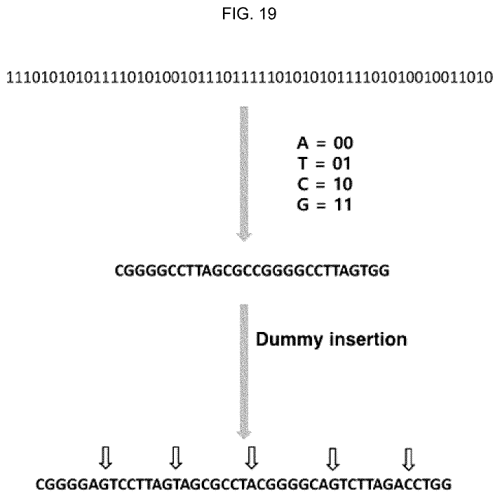

FIG. 19 is an exemplary diagram illustrating a DNA encoding method according to another embodiment of the present specification.

Referring to FIG. 19, it can be seen that dummy bases composed of three-base sequences such as ‘AGT’, ‘GTA’, ‘TAC’, ‘AGT’, and ‘ACC’ have been added. The number of bases forming the dummy base information may be set variably. In addition, the bases located at both ends of the added dummy base information may be different from the adjacent bases in the first-converted base sequence information. This is also intended to prevent the same base from being synthesized repeatedly.

Furthermore, the examples shown in FIGS. 17 and 19 illustrate cases where dummy bases are inserted within the first-converted base sequence to perform the second conversion. In such cases, the bases located inside the DNA molecule are bonded to other bases on both sides. However, a base located at the very end of the DNA molecule is connected on only one side, and has no molecular bonding on the other side. In such a case, there is a possibility that the base at the end may become disconnected and lost. Since a base corresponding to actual data located at the end may be damaged, it is also necessary to provide protection for such end portions.

Referring again to FIG. 16, in step S300 following step S200, the processor may perform a third conversion by adding protective dummy base information to both terminal ends of the second-converted base sequence information.

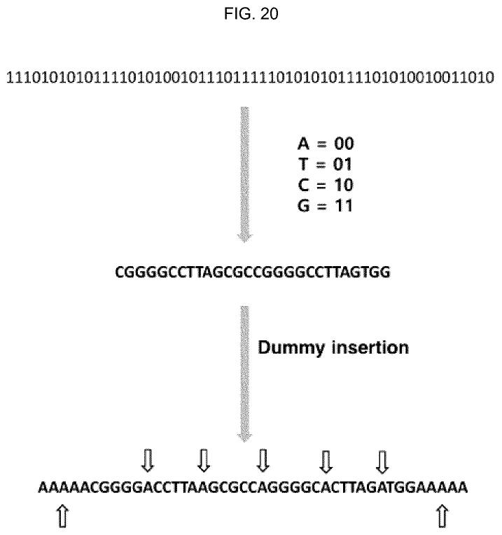

FIG. 20 is an exemplary diagram illustrating a DNA encoding method according to still another embodiment of the present specification.

Referring to FIG. 20, it can be seen that protective dummy bases composed of ‘AAAAA’ are added to both terminal ends of the second-converted base sequence information shown in FIG. 17. Since the protective dummy bases added to both ends are bases unrelated to actual information, even if they are lost, the actual data can be protected from damage.

Meanwhile, the processor may include a microprocessor, an application-specific integrated circuit (ASIC), another chipset, logic circuits, registers, communication modems, data processing devices, or the like known in the technical field of the present invention, in order to perform the above-described calculations and various control logic. In addition, when the above control logic is implemented in software, the processor may be implemented as a set of program modules. In this case, the program modules may be stored in a memory device and executed by the processor.

The above-described computer program may include code written in a computer language such as C/C++, C#, JAVA, Python, or machine language, which is readable through the device interface of the computer, so that the computer can execute the above-described methods implemented as a program. Such code may include functional code related to functions such as functions defining the necessary operations to perform the methods, as well as control code related to execution procedures necessary for the processor of the computer to execute the functions in a predetermined sequence. In addition, the code may further include memory reference-related code that indicates which location (address) in the internal or external memory of the computer should be referenced for additional information or media required by the processor to execute the functions. Furthermore, when the processor of the computer needs to communicate with another computer or server located remotely in order to execute the functions, the code may further include communication-related code that defines how to communicate with such a remote computer or server using the communication module of the computer, and what information or media is to be transmitted and received during such communication.

The storage medium referred to herein does not denote a medium that stores data only momentarily, such as a register, cache, or memory, but rather refers to a medium that stores data semi-permanently and allows reading by a device. Specifically, examples of such a storage medium include, but are not limited to, ROM, RAM, CD-ROMs, magnetic tapes, floppy disks, and optical data storage devices. That is, the program may be stored in various recording media on various servers accessible by the computer, or on various recording media within the user's computer. Furthermore, the medium may be distributed across computer systems connected through a network, and code readable by a computer may be stored in a distributed manner.

While exemplary embodiments of the present specification have been described above with reference to the accompanying drawings, it will be understood by those skilled in the art that the present invention may be implemented in other specific forms without departing from the technical spirit or essential characteristics of the invention. Therefore, the above-described embodiments are to be understood in all respects as illustrative and not restrictive.

1. a DNA encoding method comprising:
(a) a step in which a processor performs a primary conversion of binary data into a base sequence;
(b) a step in which the processor finds a point where the same base is repeated within the primarily converted base sequence; and
(c) a step in which the processor performs a secondary conversion into a base sequence to which a dummy base is added at the point where the same base is repeated.

2. The method of claim 1,
the step (b) is a step in which the processor finds a point where the same base is repeated a predetermined number of times.

3. The method of claim 2,
the step (b) is a step in which the processor finds a point where the same base is repeated according to the number set for each base.

4. The method of claim 1,
the step (b) comprising:
(b-1) a step in which the processor generates a frequency table for repeated bases within the primarily converted base sequence;
(b-2) a step in which the processor determines the number of repetitions of the same base that requires addition of a dummy base, using the generated frequency table; and
(b-3) a step in which the processor finds a point where the same base is repeated within the primarily converted base sequence according to the number determined in the step (b-2).

5. The method of claim 4,
the step (b-2) is a step in which the processor determines the average value of the frequency as the number of repetitions of the same base that requires a dummy base.

6. The method of claim 5,
the step (b-2) is a step in which the processor determines, for each base, the average value of the frequency as the number of repetitions of the same base that requires a dummy base for each base.

7. The method of claim 1,
the step (c) is a step in which the processor performs the secondary conversion by adding at least one or more dummy bases having a predetermined sequence.

8. The method of claim 1,
the step (c) is a step in which the processor adds, as a dummy base, a base different from the bases adjacent on both sides of the point where the same base is repeated, and performs the secondary conversion.

9. The method of claim 8,
the step (c) is a step in which the processor performs the secondary conversion by adding a dummy base having a sequence of 2 or more.

10. A computer program recorded in a computer-readable recording medium, written so that each step of the DNA encoding method is performed in a computer,
the DNA encoding method comprising:
(a) a step in which a processor performs a primary conversion of binary data into a base sequence;
(b) a step in which the processor finds a point where the same base is repeated within the primarily converted base sequence; and
(c) a step in which the processor performs a secondary conversion into a base sequence to which a dummy base is added at the point where the same base is repeated.

11. The computer program recorded in a computer-readable recording medium of claim 10,
the step (b) is a step in which the processor finds a point where the same base is repeated a predetermined number of times.

12. The computer program recorded in a computer-readable recording medium of claim 11,
the step (b) is a step in which the processor finds a point where the same base is repeated according to the number set for each base.

13. The computer program recorded in a computer-readable recording medium of claim 10, the step (b) comprising:
(b-1) a step in which the processor generates a frequency table for repeated bases within the primarily converted base sequence;
(b-2) a step in which the processor determines the number of repetitions of the same base that requires addition of a dummy base, using the generated frequency table; and
(b-3) a step in which the processor finds a point where the same base is repeated within the primarily converted base sequence according to the number determined in the step (b-2).

14. The computer program recorded in a computer-readable recording medium of claim 13,
the step (b-2) is a step in which the processor determines the average value of the frequency as the number of repetitions of the same base that requires a dummy base.

15. The computer program recorded in a computer-readable recording medium of claim 14,
the step (b-2) is a step in which the processor determines, for each base, the average value of the frequency as the number of repetitions of the same base that requires a dummy base for each base.

16. The computer program recorded in a computer-readable recording medium of claim 15,
the step (c) is a step in which the processor performs the secondary conversion by adding at least one or more dummy bases having a predetermined sequence.

17. The computer program recorded in a computer-readable recording medium of claim 10,
the step (c) is a step in which the processor adds, as a dummy base, a base different from the bases adjacent on both sides of the point where the same base is repeated, and performs the secondary conversion.

18. The computer program recorded in a computer-readable recording medium of claim 17,
the step (c) is a step in which the processor performs the secondary conversion by adding a dummy base having a sequence of 2 or more.
